(19) United States
(12) Patent Application Publication
(10) Pub. No.: US 2015/0209190 A1
(43) Pub. Date: Jul. 30, 2015

(54) METHOD AND APPARATUS FOR CUTTING DISPOSABLE ABSORBENT SANITARY ARTICLES OF THE PRECLOSED TYPE THAT CAN BE WORN LIKE PANTS

(71) Applicant: Fameccanica.Data S.P.A., Pescara (IT)

(72) Inventors: Gabriele Sablone, Montesilvano (Pescara) (IT); Cristian Giuliani, Pineto (Teramo) (IT)

(21) Appl. No.: 14/381,071

(22) PCT Filed: Mar. 1, 2013
(86) PCT No.: PCT/IB2013/051657

§ 371 Date: Aug. 26, 2014

(30) Foreign Application Priority Data
Mar. 9, 2012 (IT) TO2012A000209

(51) Int. Cl. A61F 13/15; B32B 38/00; B32B 38/18; B32B 37/18

(52) U.S. Cl. CPC A61F 13/15739; A61F 13/15764; B32B 37/18; B32B 38/0004; B32B 38/0012; A61F 13/15699; B32B 38/18; B32B 2307/726; B32B 2555/02

(57) ABSTRACT

A device and method for producing sanitary articles of the pre-closed pant type (10) comprising the steps of: folding longitudinally in two a composite web (100), formed by a chain of blanks of absorbent sanitary products (10), which advance in a longitudinal direction (A), so as to form a continuous longitudinal portion of web and a discontinuous portion of web, the continuous portion comprising the waist regions, the discontinuous portion being basically constituted by the absorbent insert; welding (40) the continuous portion (38) of the web (100) with welding lines (13) transverse to the longitudinal direction of advance (A) so as to form the circumference of the waist and the openings for the legs (11); of the sanitary article (10); conveying (50) the composite web (100) through the welding station (40), maintaining a positive grip of the discontinuous portions (39) of said composite web (100); feeding the cutting station (60) with said composite web (100), maintaining the positive grip of the discontinuous portions (39) of said composite web (100); and cutting said composite web (100) to separate the individual sanitary articles (10) from one another.

### TECHNICAL FIELD

The present description relates to a cutting device that can be used in the production of sanitary articles, such as, for example, nappy-pant products of the pre-closed type.

The reference to this specific field of application must not, on the other hand, be interpreted in a sense that limits the scope of the invention.

### TECHNOLOGICAL BACKGROUND

A common technique for the production of sanitary products, of the pre-closed or pre-fastened type, that can be worn as pants, normally referred to as “training pant” or “pull-ups”, consists in providing a composite web formed by a continuous chain of blanks of product aligned so as to have the transverse axis of each individual absorbent article coinciding with the direction of movement of the web itself. The continuous composite web is folded in two about said transverse axis so as to set the opposite longitudinal edges of the web on top of one another. The two flaps of the composite web thus set on top of one another are connected together with welding lines so as to define the openings for the waist and for the legs of each absorbent article. The continuous chain of blanks, at the welds, is subsequently subjected to a cutting operation to divide the finished products from one another.

This particular methodology of production is normally identified with the term “cross direction” (or CD) to distinguish it from the more traditional form of production, referred to as “machine direction” (or MD), which forms the absorbent articles while they travel with their own longitudinal axis parallel to the direction of production.

An example of a method of production of absorbent sanitary products of the CD type is described in the documents Nos. EP-A-1 013 251 and IT-0001379452, filed in the name of the present applicant.

In CD methods, it is necessary to ensure proper positioning of the transverse welds of each product so as to define properly the openings for the waist and for the legs of each individual nappy. Moreover, it is important for the cutting line envisaged for dividing the individual products from one another to be properly positioned at the welds made between two adjacent absorbent products and it is even more important for both the welds and the cuts to present a constant angular position with respect to the direction of advance of the web; i.e., in other words, at least the cuts must always present the same angular relation with respect to the edges of the composite web.

According to the known art, to obtain sanitary articles with the welds and the cuts in a proper phase relation with respect to one another and with an angular relation with respect to the edges of the article constant in time, there becomes necessary extremely complex and costly equipment, where said operations of welding and cutting are performed on a single roller run over which is the continuous web made up of the blanks of the individual absorbent articles.

It is obvious to the person skilled in the branch that a solution of this sort brings with it problems that are difficult to solve, such as for example the complexity of the apparatus and the low flexibility in the change of the format of the articles, with the consequent rise in the operating costs. The low flexibility to the change of format is basically due to the fact that the diameter of the roller must be chosen so that the circumference is an integer multiple of the transverse development of the absorbent product that is being produced. Consequently, as the article changes, the transverse development of the absorbent article changes, and accordingly the diameter of the roller changes. As a result, the entire welding and cutting unit must be replaced.

The complexity of the known equipment derives from the fact that the cutting and welding tools must be accommodated around a single roller that functions as contrast roller both for the welding operation and the cutting operation. Consequently, said roller must be of large diameter to be able to house on its periphery both the welding mechanisms and the cutting devices and, on account of the delicacy of both processes, said roller must present an equally high precision that raises the costs thereof.

The foregoing is aggravated by the fact that to be able to carry out a weld of adequate mechanical characteristics it is necessary to heat the composite web formed by the continuous chain of blanks. For this purpose, normally said heating is obtained by inserting resistances in the areas that are to be welded, which are obviously also cutting areas. On account of the large diameter of the contrast roller, and hence on account of the resulting considerable mass, management of the temperature of said contrast areas presents problems that are difficult to solve and lead to the reduction of the working life of the welding and cutting tools, which, inevitably, are affected by the variations in temperature and hence by the consequent dimensional variations of the common contrast element.

### OBJECT AND SUMMARY

The object of the present invention is to improve the technique for the production of sanitary articles of the pre-closed type that can be worn as pants so that the opposite transverse edges of each article always maintain the proper relation of shape and phase with the corresponding transverse welding line.

According to the present invention, said object is achieved by equipment and by a method forming the subject of Claims 1 and 7.

The claims form an integral part of the technical teaching provided herein in relation to the invention.

### BRIEF DESCRIPTION OF THE DRAWINGS

The invention will now be described, purely by way of non-limiting example, with reference to the annexed drawings, in which:

FIG. 1 is a perspective view of an absorbent product of the pre-closed pant type that can be obtained with the apparatus according to a preferred embodiment of the present invention, in a closed configuration ready for use;

FIG. 2 is a schematic perspective view of the absorbent product of FIG. 1 in the splayed-out configuration;

FIG. 3 is a schematic perspective view of the steps of production of an absorbent article of the type represented in FIGS. 1 and 2;

FIGS. 4 and 5 are schematic perspective views of equipment for producing the absorbent articles according to a preferred embodiment of the present invention;

FIG. 6 is a schematic perspective view of the detail regarding the welding equipment according to a preferred embodiment of the present invention;

FIG. 7 is a schematic illustration of the characteristics of welding and cutting in articles obtained according to a preferred embodiment described herein; and

FIG. 8 illustrates in side view the detail regarding the cutting device according to a preferred embodiment of the present invention.

### DETAILED DESCRIPTION OF THE EMBODIMENTS

Illustrated in the ensuing description are various specific details aimed at providing an in-depth understanding of the embodiments. The embodiments may be obtained without one or more of the specific details, or with other methods, components, materials, etc. In other cases, known structures, materials, or operations are not illustrated or described in detail so that various aspects of the embodiments will not be obscured.

Reference to “an embodiment” or “one embodiment” in the framework of the present description is intended to indicate that a particular configuration, structure, or characteristic described in relation to the embodiment is comprised in at least one embodiment. Hence, phrases such as “in an embodiment” or “in one embodiment” that may be present in various points of the present description do not necessarily refer to one and the same embodiment. Moreover, particular conformations, structures, or characteristics may be adequately combined in one or more embodiments.

The references used herein are provided merely for convenience and hence do not define the sphere of protection or the scope of the embodiments. Consequently, the definitions “front” or “rear” referring to the absorbent sanitary article depend only upon the final use of the product itself, i.e., upon how it is worn by the user, whereas the definitions “top” or “bottom” regarding possible elements present in one or more embodiments described depend only upon the viewing point of the observer with respect to the drawings annexed to the present document, without thereby limiting the principle and/or the scope of the invention.

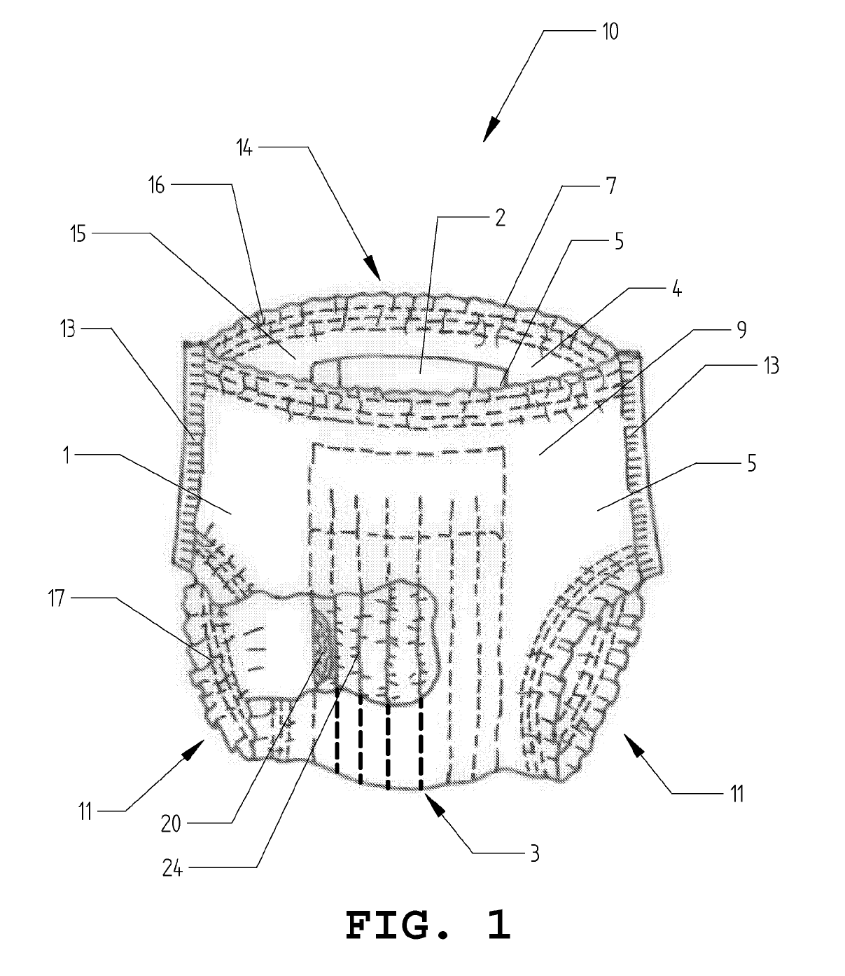

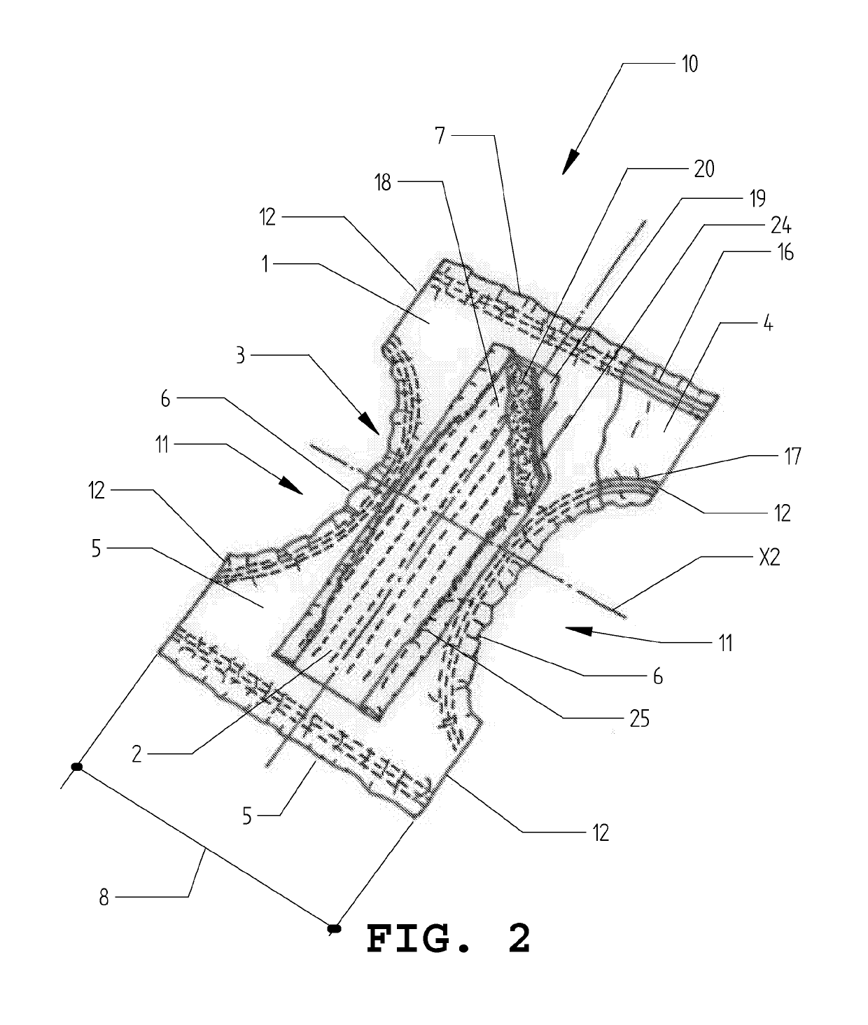

With reference to FIGS. 1 and 2, designated by 10 is a disposable absorbent product of the so-called “pull-on” or “training-pant” type, obtained with a method and an apparatus in accordance with the teachings provided by the present invention.

In FIG. 1, the absorbent article is represented in its closed condition, where it has a conformation substantially resembling that of a pair of pants, whereas FIG. 2 illustrates the same product as that of FIG. 1 in the splayed-out or open configuration, i.e., before it is folded about its transverse axis X2 and before the welds 13 that bestow upon it the characteristic pant-like conformation with definition of the opening for the waist 14 and the two openings for the legs 11 have been made.

In basic terms, the product 10 is made up of a chassis 1, or outer part of the absorbent product, which, during use of the sanitary article, supports the absorbent insert 2 and keeps it in contact with the body of the user.

Nappies of this sort may present shapes and structures different from one another; consequently, the shape and structure of the absorbent nappy that is described in the sequel of the present document is to be understood merely by way of example, to enable a better appreciation of the advantages of the present invention.

The absorbent product represented in FIGS. 1 and 2 has the chassis 1 that can be obtained from an outer sheet 4 and by an inner sheet 5, between which there can be interposed elastic elements 16 and 17 that contribute to enhancing the characteristics of wearability thereof.

The elastic elements 16 extend along the front waist edge 5 and rear waist edge 7 and are designed to elasticate the opening for the waist 14. The elastic elements 17 are designed to elasticate the openings for the legs 11.

The two sheets 4 and 5 constituting the chassis 1 are normally made of non-woven fabrics that may have undergone treatments that render them impermeable to liquids so as to contain any possible leakage of liquids and prevent any possible contamination of the clothes of the user with which the chassis 1 is in contact.

The elastic materials 16 and 17, in turn, can be chosen from among the multiplicity of materials available on the market, such as, for example, strips made of synthetic rubber, manufactured by Fulflex, or yarn made of Lycra®, manufactured by Invista.

In the absorbent product, represented in FIGS. 1 and 2, located on the chassis 1, in the groin region 3, set between the two waist regions, the front one 9 and the rear one 15, and the openings for the legs 11, is the absorbent insert 2 that is designed to come into contact with the body of the user, assuming the general U shape. The function of the absorbent insert 2 is that of collecting and withholding the body fluids.

One of the functions of the elastic elements 16 and 17 of the chassis 1 described above is that of keeping the absorbent insert 2 in the proper working position, even when it gets heavy because it has absorbed the body fluids.

The two sheets 4 and 5 constituting the chassis 1 and said elastic materials 16 and 17 are rendered fixed by means of adhesives and/or thanks to welds performed with heated rollers or ultrasomic systems.

The absorbent insert 2 has a structure in which there may be recognized (in addition to various other accessory elements):
a top layer or topsheet 18 permeable to evacuated body liquids, designed to face the body of the user;
a bottom layer or backsheet 19 impermeable to body liquids and designed to face the outside, i.e., the chassis 1; and
an absorbent core 20 set between the topsheet 18 and the backsheet 19 designed to absorb and withhold exudates.

On the absorbent product 10, and in particular on the absorbent insert 2, there may be present other characteristic elements that contribute to enhancing the characteristics of wearability and absorbance thereof, such as for example the so-called “cuffs” or elasticated sides 25 set at the sides of the absorbent core 20 with the function of lateral containment of body fluids.

For a more detailed description of the elements listed above and of other elements, well known to persons skilled in the art, the reader is referred to the extensive literature existing on the subject, such as for example the U.S. Pat. No. 4,704,116, granted on Nov. 3, 1987 to Enloe.

In a possible embodiment, as highlighted in FIGS. 1 and 2, the absorbent insert 2 and the chassis 1 are rendered fixed with respect to one another by means of adhesive strips 24 and/or by means of welds obtained with any system in itself known, such as for example mechanical or ultrasonic heat sealing.

As highlighted in FIG. 2, moreover, on the chassis 1 there is normally made a cut 6 of appropriate shape that defines and delineates the contour of the openings for the legs 11.

As is evident to the person skilled in the art, the representations of FIGS. 1 and 2 are of a schematic nature and intend to highlight that the method and the apparatus forming the subject of the present invention may be applied to a wide variety of possible types of the absorbent product 10.

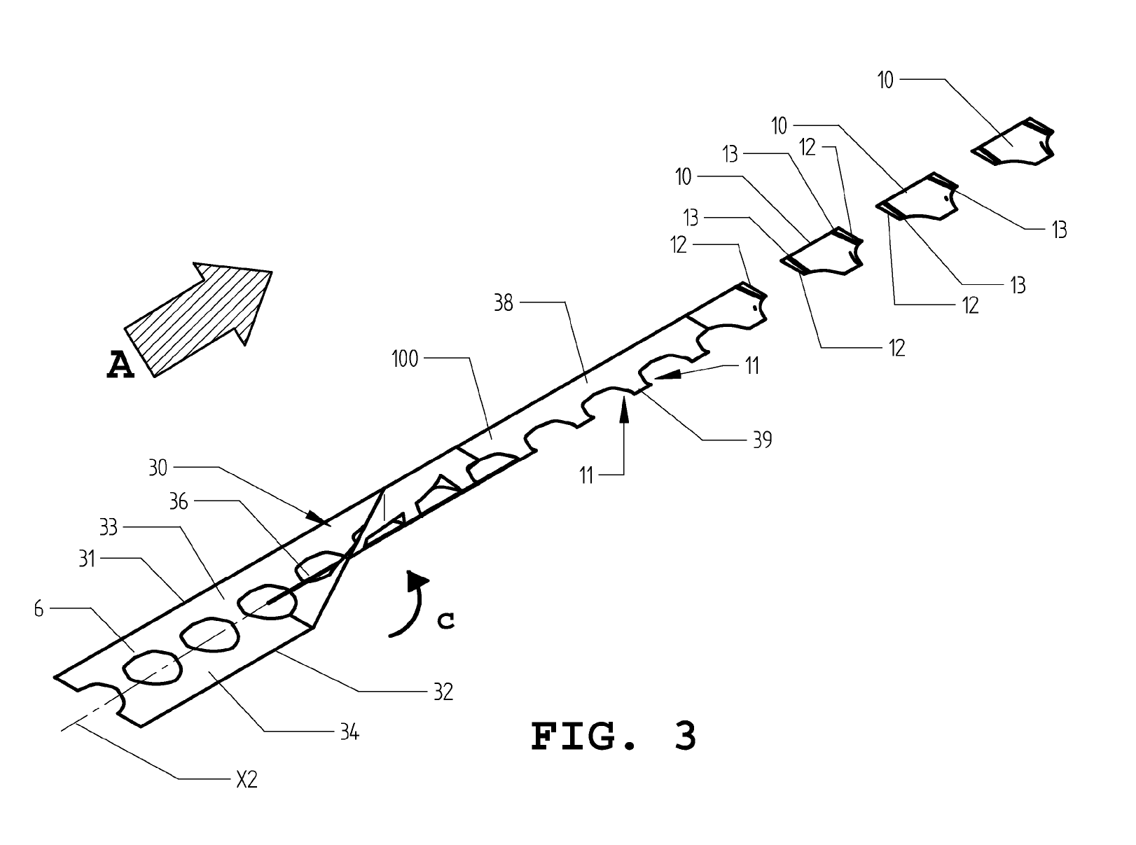

Schematically represented in FIG. 3 is part of the method of production of an absorbent sanitary article 10. Designated by 100 in FIG. 3 is a composite web, formed by a chain of blanks of absorbent sanitary products 10, which advance continuously in a longitudinal direction A. The composite web 100 has two opposite longitudinal ends 31 and 32, which on the product provide, respectively, the front waist edge 5 and the rear waist edge 7. Said edges, during the process of manufacture of the absorbent sanitary article, are set on top of one another following upon a fold around the bar 36 parallel to the longitudinal direction of advance A of the web 100 and coinciding, normally, with the axis of symmetry X2 of the individual absorbent products 10, in the longitudinal folding station 30, of which the bar 36 is an essential component. The arrow C indicates schematically the movement of folding that leads to obtaining superposition of the longitudinal edges 31 and 32. After the fold about the longitudinal axis X2 the front flap 33 and the rear flap 34 of the web 100, respectively connected at the longitudinal edges 31 and 32, are joined together at regular intervals, as illustrated in the absorbent article of the pre-closed pant type of FIG. 1, which can be obtained with the apparatus according to the embodiment of the present invention exemplified herein, it being possible for said connection to be obtained by means of the two welds 13.

An example of equipment and of method 30 suitable for making a fold of this sort is described in the document No. TO2011A001085, filed in the name of the present applicant.

After the operations of folding and closing, the continuous composite web is subjected to operations of cutting along the transverse lines 12 to form the individual product 10.

It will be appreciated from FIGS. 1 and 2 what may be the complexity of the web that is generated in a production method in which the blanks of products 10 extend in a direction transverse to the direction of movement. It will moreover be appreciated that the structure of the web and its management become even more complex once it is folded in two. In fact, on account of superposition of the sheet, the differences of thickness both in the transverse direction and in the longitudinal direction of the web 100 are considerably amplified and problems are generated in conveyance of the web 100 on account of its discontinuity generated by the cuts 6 for providing the openings for the legs 11 present on each article 10. In fact, as is clearly illustrated in FIG. 3, the web 100 has a continuous longitudinal portion (or band) 38 that coincides with the front waist region 9 and rear waist region 15, and a discontinuous longitudinal portion (or band) 39 that coincides with the portion that comprises the groin region 3 set between the front waist region 9 and rear waist region 15 and the openings for the legs 11.

From what has been said, it will hence be appreciated how problematical it is to ensure that during the operations of welding and cutting of each product 10 there is always kept an accurate phase relation between the transverse welds 13 and the cuts 12, as well as a constant angular relation between said transverse welds 13 and the cuts 12 with respect to the waist edges 5 and 7 of each absorbent article 10.

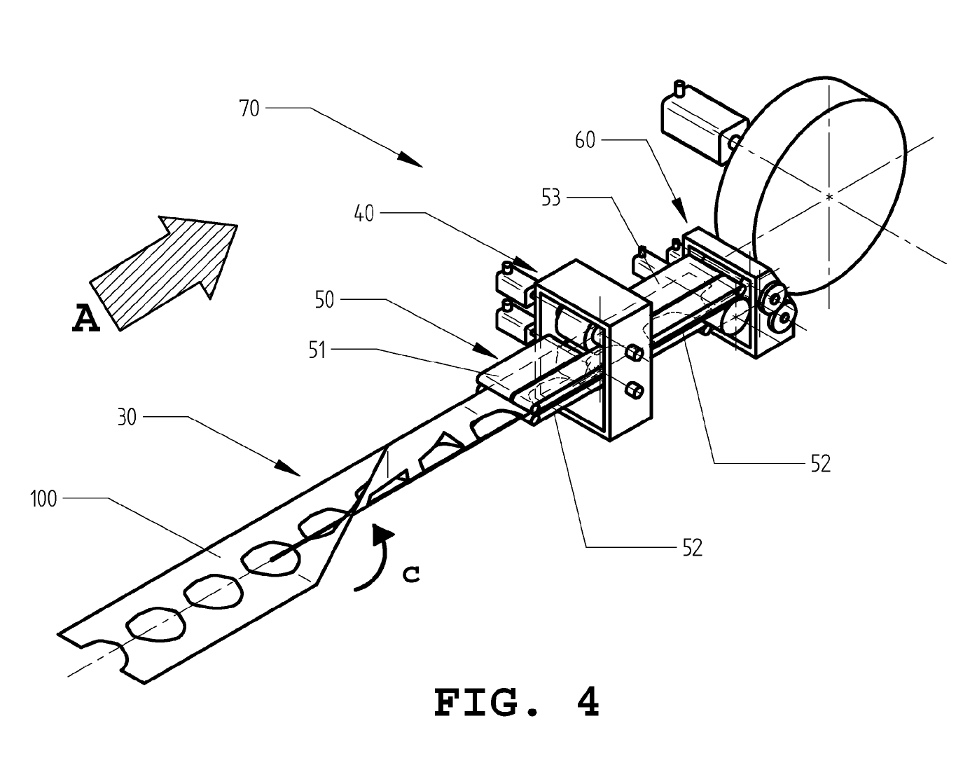

Schematically illustrated in FIG. 4 is the apparatus 70 according to one embodiment of the present invention.

In the embodiment exemplified, the apparatus 70 is made up of the equipment 40, which is able to provide transverse welding lines 13, designed to define the waistline and the openings for the legs in each pre-closed absorbent sanitary article 10 of the pant type, such as for example the one represented in FIGS. 1 and 2.

In the preferred embodiment, as illustrated in FIG. 5, the transverse station 40 may be made up of a structure 41 where a pair of counter-rotating rollers is installed, designed to perform, one the function of welding roller 42 and the other the function of contrast roller or counter-welding roller 43.

The web of blanks 100 is made to pass, in the longitudinal direction of advance A, between the welding roller 42 and the counter-welding roller 43, which turn so as to have the respective tangential speeds substantially identical to one another and equal to the speed of linear advance of the chain of blanks 100.

The welding roller 42 and counter-welding roller 43 are driven in a known way, for example by a single motor drive with the provision of transmission means, or else, according to a currently preferred solution, illustrated in FIGS. 4, 5 and 6, by means of two independent motor drives constituted by two servo motors 44 and 45 controlled electronically with respect of one another, each mechanically connected by means of joints, preferably of the constant-velocity joint type 440, to the respective roller.

In the device described herein, the action of welding is obtained with the combined application of pressure and heat. For this purpose, both of the rollers carry mounted within them (according to a configuration in itself known and used for example in “flow-pack” packaging machines) heating resistors and means for controlling the temperature.

The pressure between the two rollers is guaranteed by means in themselves known, such as for example pneumatic, or hydraulic, or mechanical systems, which are well known by persons skilled in the field and are not illustrated in the figures for simplicity of representation.

The roller 42 that has the function of welding roller is provided along its periphery with projecting welding blades 46a and 46b, transverse to the direction of advance of the composite web 100, in other words, substantially aligned with the axis of rotation X4 of the welding roller itself 42. In the preferred embodiments illustrated in the present document, the welding blades 46a and 46b form with any generatrix of the cylinder of the welding roller parallel to the axis of rotation X4 an angle of 3° to 4°.

The roller 43 that has the function of contrast roller has, instead, a completely smooth surface of the mantle.

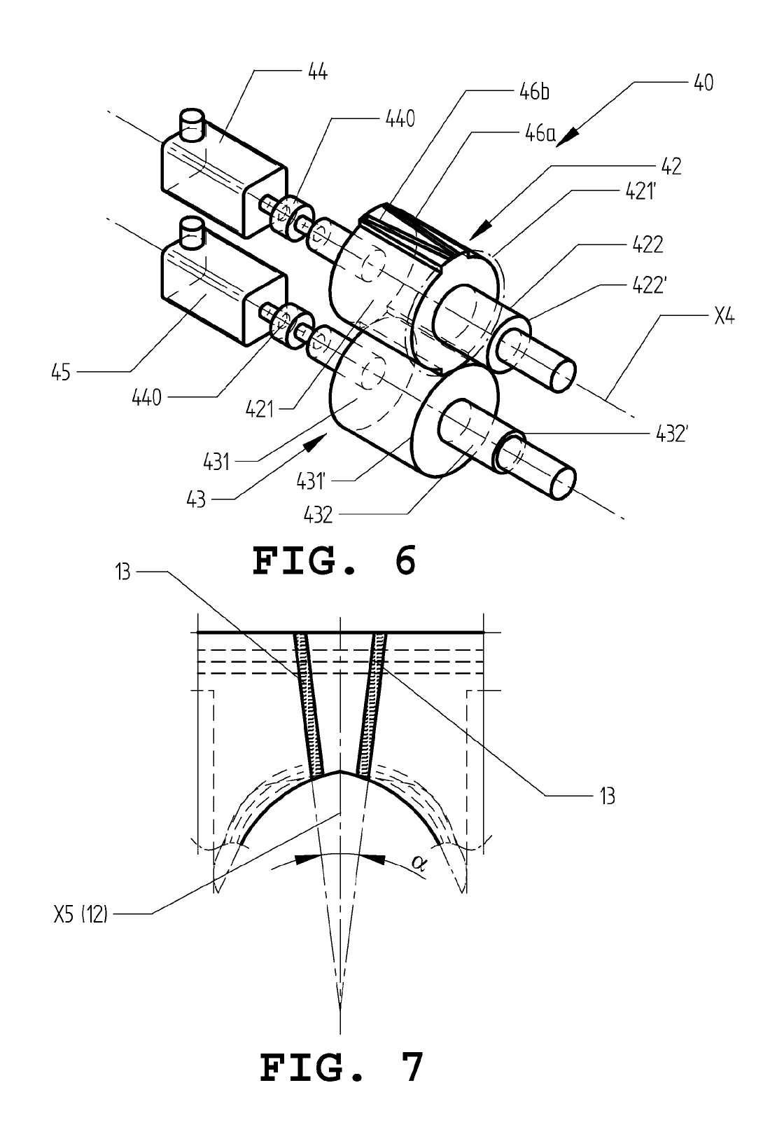

FIG. 7 of the annexed drawings represents the end regions of two mutually adjacent sanitary articles 10 still connected in the production chain 100, with highlighted the two welding lines 13 and, set between them, the dashed and dotted line X5 that coincides with the line of separation 12 between the two adjacent articles 10, which will be obtained with the next cutting operation.

As is known, and as is clearly highlighted in FIG. 7, the welding lines 13 may present a slight convergence with respect to one another, such as to form a maximum angle α of approximately 6° to 8°.

In the preferred embodiment, as illustrated in FIG. 4, the composite′ web 100 at output from the folding unit is taken up by a conveying system 50, which is in turn constituted by:
a first conveying means (or conveyor) 51, designed to pick up at output from the folder 30 the continuous part of the composite web 100 and feed it to the welding rollers 42 and 43; as is well known, the continuous band 38 of the web 100 has a small thickness, basically consisting of the two flaps 33 and 34 set on top of one another;
a second conveying means (or conveyor) 52, designed to convey the discontinuous band 39 of the web 100 from the folder 30 as far as the cutting assembly 60 traversing the welding unit; and
a third conveying means (or conveyor) 53 that picks up the continuous band 38 of the web 100 leaving the welding rollers 42 and 43 and feeds it to the cutting assembly 60.

In the preferred embodiment, as highlighted more clearly in FIG. 5, the first conveyor 51 may be provided by a top carriage 54 and by a bottom carriage 55. Both of the carriages are equipped with appropriate motor means (not illustrated) capable of bestowing on the web 541 of the top carriage 54 and the web 551 of the bottom carriage 55 a linear speed of advance equal to that of the web 100. The two carriages 54 and 55 are provided with mechanical means, in themselves known, which are able to grip the continuous flaps 33 and 34 of the web 100 between them. Moreover, in order to increase the gripping capacity of the chain of blanks 100 exerted by said carriages 54 and 55, one or both of the belts 541 and 551 may be of the “suction” or “vacuum” type; i.e., they may have the surface permeable to air, which, being connected to a source of subatmospheric pressure, with means in themselves known, is able to increase the overall gripping force that can be applied to the web 100 by the conveyor 51.

In the preferred embodiment, also the second conveyor 52 may be provided by a top carriage 56 and a bottom carriage 57. Both of the carriages are equipped with appropriate motor means 562 that are able to bestow on the belts 561 and 571 of the two respective carriages 56 and 57 a linear speed of advance equal to that of the web 100 and are able in turn to keep the discontinuous parts 39 of the web 100 gripped together thanks to mechanical means in themselves known.

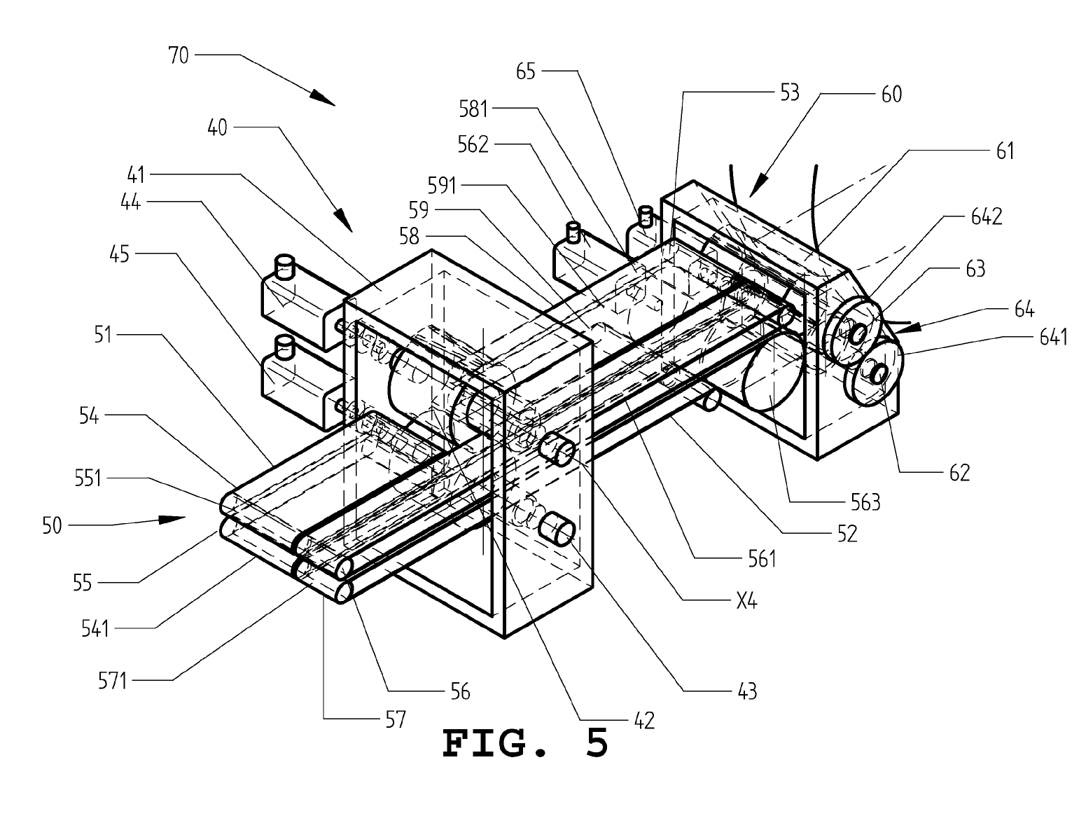

In order to increase the gripping capacity of the discontinuous parts of the web 100, also in this case, one or both of the belts 561 and 571 belonging, respectively, to the top carriage 56 and to the bottom carriage 57 of the second conveyor 52, may be of the “suction” or “vacuum” type; i.e., they may have the surface permeable to air, which, being connected to a source of subatmospheric pressure with means and techniques well known to persons skilled in the sector, increases the overall gripping force that the conveyor 52 is able to apply to the web 100. The conveying system 52 grips the discontinuous band 39 of the web 100 leaving the folding system 30 and accompanies it as far as the cutting assembly 60, traversing the welding station 40 without ever abandoning the discontinuous band 39 of the web 100. Traversal of the welding station 40 by the second conveyor 52, as is clearly evident in FIGS. 5 and 6, is made possible by the fact that the welding roller 42 and counter-welding roller 43 have, respectively, a second portion 422 and a second portion 432 with corresponding diameters 422′ and 432′ smaller than the diameters 421′ and 431′, respectively, of the first working portion 421 of the welding roller 42 and of the first working portion 431 of the counter-welding roller 43. As illustrated in FIG. 5, the conveying means 52 manages to traverse the welding unit 40 in the space that is created between the two second portions of welding roller 422 and counter-welding roller 432 of diameter smaller than the corresponding nominal working diameters of the first portions 421 and 431.

In the preferred embodiment, the third conveyor 53 may be provided by a top carriage 58 and a bottom carriage 59. Both of the carriages are equipped with appropriate motor means 562, which are in common with the second conveying system 52 and are able to bestow on the web 581 of the top carriage 58 and the web 591 of the bottom carriage 59 a linear speed of advance equal to that of the web 100. Also the two carriages 58 and 59 are provided with mechanical means, in themselves known, which are able to grip the continuous band 38 of the web 100 between them. Moreover, in order to increase the gripping capacity of the chain of blanks 100 provided by said carriages 58 and 59, one or both of the belts 581 and 591 may be of the “suction” or “vacuum” type; i.e., they may have the surface permeable to air, which, by being connected to a source of subatmospheric pressure with means in themselves known, is able to increase the overall gripping force that can be applied to the continuous band 38 of the web 100 by the conveyor 53.

In the preferred embodiment, the two conveying systems 52 and 53, as illustrated in FIG. 4, have the purpose of feeding the web 100, made up of the chain of blanks of absorbent articles 10, to the cutting station 60. For this purpose, the bottom carriage 57 of the second conveying system 52 and the bottom carriage 59 of the third conveying system 53 may have the drive roller 563 in common. In the embodiment described herein, the drive roller 563 is set in the proximity of the cutting station 60 and feeds it with the composite web 100.

The cutting station 60 may comprise a supporting structure 61, on which the cutting (or blade) roller 62 and the contrast (or counterblade) roller 63 are mounted. The blade roller 62 and counterblade roller 63 may be motor-driven by a drive means 65 that in the preferred configuration may be constituted by an electronically controlled servo motor, which, in turn, may be mechanically connected to the cutting or blade roller 62 by means of an appropriate constant-velocity joint. Furthermore, the blade roller 62 drives the counterblade roller 63 by means of a system of transmission of motion that may be of the gear type 64, where the gears, i.e., the drive gear 641 and driven gear 642, are fitted, respectively, on the blade roller 62 and on the counterblade roller 63.

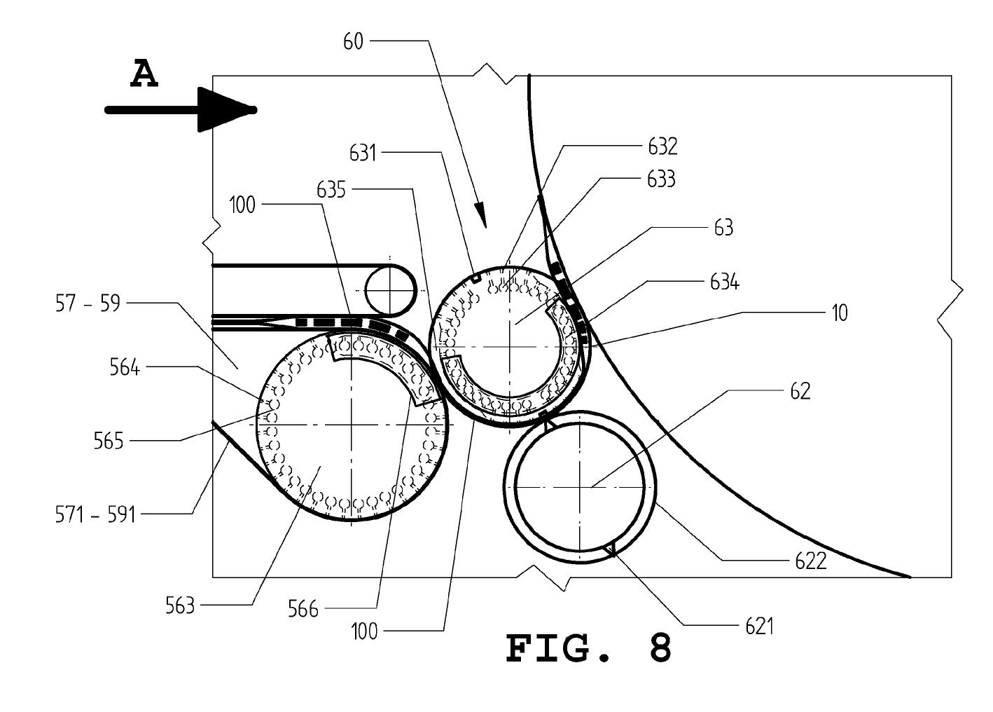

As illustrated more clearly in FIG. 8, in the preferred embodiment, the blade roller carries on its surface at least one cutting blade 621.

Each blade 621 co-operates with the counterblade roller 63 on a corresponding contrast insert 631.

As is known, the blade 621 and the counterblade roller 63, in particular the contrast insert 631, must have peripheral speeds that are substantially the same as one another and equal to the speed of feed of the composite web 100.

Consequently, the diameter of the counterblade roller 63 must be chosen so as to have a development of the circumference equal to an integer multiple of the transverse development 8 of the product 10 of FIG. 2 in the splayed-out condition.

Obviously, what has been said makes it clear that the cutting station 60 is one of those components of the production line that must undergo the operations of change of format when it is decided to produce an absorbent article of different dimensions (change of size).

As may be appreciated from the representation of FIG. 8, during the step of feed of the cutting station 60, the composite web 100 is stably held, without ever being released; in fact, both the continuous band 38 and the discontinuous band 39 of said web 100, passing from said roller 563 of the conveying means 50 to said counterblade roller 63 of said cutting unit 60 follow a curvilinear path before the continuous band 38 of said web 100 is subjected to the action of cutting by the blade 621.

According to a preferred embodiment, as illustrated in FIG. 8, the action of gripping of the composite web 100 may be further improved by getting said web 100, both the longitudinal continuous portion (or band) 38 and the discontinuous longitudinal portion (or band) 39 of the web 100, to describe an S-shaped path.

The action of gripping of the composite web 100 may be rendered even more effective by causing the permeable surfaces of the belts 571 and 591 to be connected to a source of subatmospheric pressure also in an area corresponding to the drive roller 563 and causing also the surface of the counterblade roller 63 to be able to exert an action of suction. Said suction action is provided in selected sectors of the circumference of the two rollers 563 and 63, as illustrated in FIG. 8. In this way, the two rollers, i.e., the drive roller 563 and the counterblade roller 63, manage to grip, respectively, the bottom surface and the top surface both of the continuous band 38 and of the discontinuous band 39 of the composite web 100 simultaneously.

It is possible to keep under suction a drive roller over which belts are run using, for example, the belts identified by the trademark Conducto® produced and marketed by the firm GKD-Gebr. Kufferath AG, Metallweberstrasse No. 46, 52353 Dueren, Germany. The drive roller 563, in this case, is perforated in a radial direction with holes 564 made in its mantle surface and arranged in rows that extend along the generatrices of the roller 563, each row of holes 564 coming under a respective manifold 565, which is in turn connected, by means of an appropriate distributor 566, held in a fixed position, to said source of subatmospheric pressure (not represented in the drawings).

Likewise, the counterblade roller 63 is perforated in a radial direction by holes 632 made in its mantle surface and arranged in rows that extend along the generatrices of the roller 63, each row of holes 632 coming under a respective manifold 633, which is in turn connected, by means of an appropriate distributor 634, held in a fixed position, to said source of subatmospheric pressure (not represented in the drawings). In addition, the counterblade roller 63 may present, on its surface, recesses 635, housed in which are the parts of larger thickness of the absorbent article, i.e., the parts in which the absorbent material of the insert 20 is present.

Consequently, the web 100 thus gripped thanks to the action exerted by the vacuum of the drive roller 563 and of the counterblade roller 63, is segmented by the blade 621 of the cutting roller 62, in an area corresponding to the continuous band 38, into the plurality of disposable absorbent products 10, which are in turn transferred by the counterblade roller 63 to the equipment set downstream of the cutting station 60, which carries out the further operations of longitudinal and/or transverse folding of the absorbent article, as well as counting, grouping, and packaging the products.

According to a preferred embodiment, as illustrated in FIG. 8, the cutting roller 62 may present on its surface a shell 622, normally made of thermoplastic material, which is able to grip the edge of the continuous band 38 of the web 100 that is created when the blade 621 has separated each absorbent article 10 therefrom.

Of course, without prejudice to the principle of the invention, the details of construction and the embodiments may vary, even significantly, with respect to what has been illustrated herein purely by way of non-limiting example, without thereby departing from the scope of the invention as defined by the annexed claims.

1. A device for welding and cutting a composite web formed by a chain of blanks of absorbent sanitary products (100), which advance continuously in a longitudinal direction (A), and comprising a continuous longitudinal portion of web (38), and a discontinuous longitudinal portion (39), said device comprising:
a cutting station (60), wherein said cutting station comprises a blade roller (62) located on which is a blade (621), and a counterblade roller (63) that co-operates with the blade functioning as contrast surface for the cut (631); and
conveying means (50) comprising at least one conveying system (52), designed to convey the discontinuous portion (39) of said composite web (100) and to feed said continuous portion (38) of said composite web (100) to said cutting station (60), wherein said conveying means (50) comprise at least one roller (563) set in the proximity of the counterblade roller (63) of said cutting unit (60), arranged in such a way that both the continuous longitudinal portion (38) and the discontinuous longitudinal portion (39) of said web (100) follow a curvilinear path along the outer surface of said roller (563) of the conveying means (50) and along the outer surface of said counterblade roller (63) of said cutting unit (60) before said continuous longitudinal portion (38) of said web (100) is subjected to the action of cutting by means of the blade (621).

2. The device according to claim 1, wherein the at least one conveying system (52) is connected to at least one source of subatmospheric pressure.

3. The device according to claim 1, wherein said conveying means (50) comprise a first, bottom, belt (57) and a second, top, belt (56) designed to grip between them respective mutually facing surfaces of the discontinuous longitudinal portion (39) of said composite web (100).

4. The device according to claim 1, wherein the at least one roller (563) of said conveying means (50) comprises means (564, 565) for gripping said composite web (100), said gripping means (564, 565) preferably comprising subatmospheric-pressure gripping means.

5. The device according to claim 1, wherein said counterblade roller (63) of said cutting station (60) comprises, on the outer surface, recesses (635) designed to receive the parts of larger thickness of said composite web (100, 10).

6. The device according to claim 1, wherein said counterblade roller (63) of said cutting station (60) comprises means (632, 633) for gripping said composite web (100, 10), said gripping means (632, 633) preferably comprising subatmospheric-pressure gripping means.

7. A method for the production of sanitary articles of the pre-closed pant type (10), comprising the steps of:
folding longitudinally in two a composite web (100), formed by a chain of blanks of absorbent sanitary products (10), which advance continuously in a longitudinal direction (A), said web comprising a continuous longitudinal portion (38) and a discontinuous portion (39), the continuous portion comprising the waist regions (9 and 15), and the discontinuous portion comprising the groin region (3) set between the two waist regions (9, 15) and the openings for the legs (11);
welding (40) said composite web (100) in the continuous longitudinal portion (38) with welding lines (13) transverse to the longitudinal direction of advance (A), set at a distance from one another by a pitch equal to the width (8) of said sanitary article designed to form the circumference of the waist (7, 5) and the openings for the legs (11) of the sanitary article (10);
cutting the continuous longitudinal portion (38) of said composite web (100) in a cutting station (60) comprising a blade roller (62) located on which is a blade (621), and a counterblade roller (63) that cooperates with the blade functioning as contrast surface for the cut (631), said cutting station (60) carrying out cuts (12) transverse to the longitudinal direction (A) of advance of said composite web (100), which are set at a distance from one another by a pitch equal to the width (8) of said sanitary article (10) and are designed to separate the individual sanitary articles (10) from one another, and
feeding said composite web (100) to said cutting station (60) by conveying means (50) comprising at least one conveying system (52) designed to convey the discontinuous portion (39) of said composite web (100) and to feed said continuous portion (38) of said composite web (100) to said cutting station (60), wherein said conveying means (50) comprise at least one roller (563) set in the proximity of the counterblade roller (63) of said cutting unit (60), arranged in such a way that both the continuous longitudinal portion (38) and the discontinuous longitudinal portion (39) of said web (100) follow a curvilinear path along the outer surface of said roller (563) of the conveying means (50) and along the outer surface of said counterblade roller (63) of said cutting unit (60) before said continuous longitudinal portion (38) of said web (100) is subjected to the action of cutting by mean of the blade (621).

8. The method according to claim 7, wherein, in the step of feed of the composite web (100), the discontinuous portion (39) of said web (100) is kept positively gripped by means of at least one pair of mutually facing conveying means (56 and 57).

9. The method according to claim 7, wherein, in the step of feed of the composite web (100), the discontinuous longitudinal portion (39) of said web (100) is kept positively gripped by subatmospheric-pressure gripping means.

10. The method according to claim 7, wherein, in the cutting step (60), parts of larger thickness of the discontinuous portion (39) of the composite web (100) are received and kept positively gripped by subatmospheric-pressure gripping means in respective recesses (635) present on the outer surface of said counterblade roller (63).
